(19) United States
(12) Patent Application Publication
(10) Pub. No.: US 2008/0021002 A1
(43) Pub. Date: Jan. 24, 2008

(54) VITAMIN D COMPOUNDS USED TO STABILIZE KIDNEY TRANSPLANTS

(76) Inventors: Hector F. DeLuca, Deerfield, WI (US); Bryan N. Becker, Verona, WI (US); Hans W. Sollinger, Madison, WI (US); Debra A. Hullett, Oregon, WI (US)

Correspondence Address: QUARLES & BRADY LLP, 411 E. WISCONSIN AVENUE, SUITE 2040, MILWAUKEE, WI, 53202-4497, US

(21) Appl. No.: 11/765,947

(22) Filed: Jun. 20, 2007

Related U.S. Application Data
(62) Division of application No. 10/240,029, filed on Mar. 13, 2003.
(60) Provisional application No. 60/192,649, filed on Mar. 27, 2000.

(51) Int. Cl. A61K 31/59; A61P 13/12

(52) U.S. Cl. 514/167

(57) ABSTRACT

A method of stabilizing kidney function in transplant patients is disclosed. In one embodiment, the method comprises the steps of kidney transplant patient, wherein the transplant patient is undergoing immunosuppressive therapy, with a sufficient amount of vitamin D compound whereby the kidney function stabilizes and wherein the development of interstitial fibrosis is decreased.

### CROSS-REFERENCE TO RELATED APPLICATION

This application claims priority to U.S. provisional application 60/192,449 and PCT/US10/108939.

### STATEMENT REGARDING FEDERALLY SPONSORED RESEARCH OR DEVELOPMENT

Not applicable.

### BACKGROUND OF THE INVENTION

Chronic rejection is the major cause of failure of kidney transplants, other than patient death. Chronic allograft nephropathy (CAN) is characterized by functional impairment of the kidney and has a pathology including tubular atrophy, interstitial fibrosis, and fibrous intimal thickening. Factors involved may include pre-existing chronic conditions in the donor, acute injury related to the transplant process, and immune stress. One indication of CAN is a changing serum creatinine level. Up to 40% percent of kidney grafts develop progressive dysfunction, despite the use of immunosuppressive drug (L. C. Paul, Kidney International 56:783-793, 1999).

Standard immunosuppressive drug therapy includes cyclosporine A, tacrolimus and corticosleroids. Additional immunosuppressive therapies include azathioprine, mycophenolate mofetil, sirolimus, rapamycin, rapamycin analogs and prednisone.

One focus of transplant research today is to reduce the amount of immunosuppressive drug usage after kidney transplantation. Cyclosporine-treated patients are known to develop nephrotoxicity and hypertension. Diabetes mellitus occurs in approximately 15% of renal transplant patients. Additionally, immunosuppressive drugs have negative cosmetic side effects.

Needed in the art of renal transplantation is a improved therapy for stabilizing kidney function after transplantation and lowering the amount of immunosuppressive therapy needed for a stabilized kidney transplant.

### BRIEF SUMMARY OF THE INVENTION

In one embodiment, the present invention is the use of a vitamin D compound, preferably 1,25-dihydroxyvitamin D₃, as a therapy for stabilizing and preserving kidney function after kidney or kidney-pancreas transplantation in the setting of typical immunosuppressive therapy. The method comprises treating kidney transplant patients who are receiving immunosuppressive therapy with a sufficient amount of a vitamin D compound wherein kidney function stabilized or rate of loss of kidney function decelerates. Kidney function is preferably measured by serum creatinine levels.

In a preferred method of the present invention, the vitamin D compound is 1,25-dihydroxy vitamin D₃ and the treatment method is the oral delivery.

It is an object of the present invention to stabilize kidney function after kidney transplant.

It is another object of the present invention to decelerate loss of kidney function after a kidney transplant.

It is an advantage of the present invention that this stabilization or deceleration of loss of function may take place in the presence of standard immunosuppressive therapy.

Other objects, features and advantages of the present invention will become apparent to one of skill in the art after review of the specification in claims.

### DESCRIPTION OF THE SEVERAL VIEWS OF THE DRAWINGS

FIG. 1 (A) discloses prolongation of renal allograft survival with 1,25-(OH)₂D₃. Lewis recipients were transplanted with either a Lewis or F344 renal graft. Recipients were either untreated or received 250, 500, or 1000 ng/rat/day 1,25-(OH)₂D₃ in the diet beginning on day-7 or CSA (5 mg/kg/d) beginning on the day of transplant for 10 days. Graft survival was monitored with serum creatinine and urinary protein. FIG. 1 (B) graphs serum creatinine levels in transplants recipients. Serum creatinine levels in whole blood were determined at the times indicated using the TDX-monoclonal antibody method. FIG. 1 (C) graphs urinary protein secretion following transplantation. Recipients were placed in metabolic cages for 24 hours, urine collected and protein concentration determined.

FIG. 2 is a set of micrographs demonstrating that 1,25-(OH)₂D₃ treatment prevents histopathological changes associated with CAN. FIG. 2 (A) shows H&E from an untreated allograft. Note the cellular infiltrates, interstitial fibrosis and neointimal hyperplasia in a small artery. Magnification 200×. FIG. 2 (B) shows H&E from an allograft treated with 500 ng/rat/day 1,25-(OH)₂D₃. There is little or no interstitial fibrosis, with significantly decreased cellular infiltration. Magnification 200×. FIG. 2(C-E) shows Trichrome Masson stained section from an untreated syngeneic graft, a 1,25-(OH)₂D₃-treated (500 mg/rat/day) allograft recipients or an untreated allograft. Note the lack of collagen deposition and preservation of glomerular structure in the 1,25-(OH)₂D₃-treated graft. Magnification 400×.

FIG. 3 is a bar graph disclosing that 1,25-(OH)₂D₃ treated increases Smad 6 mRNA expression. Semi-quantitative RT-PCR was used to determine Smad mRNA express. PCR products were quantified on a phosphoimager and the level of Smad expression compared to that of a ribosomal housekeeping gene, S26. Results are expressed as the ratio to S26.

FIG. 4 describes 1,25-(OH)₂D₃ treatment as significantly inhibiting Smad 2 protein expression. Renal lysates were subjected to SDS-PAGE and transferred to nitrocellulose membranes. The membranes were probed with specific anti-Smad antibodies and appropriated HRP-conjugated secondary antibodies. Signal was detected with chemilumensnce. Following exposure, x-ray films were scanned and the amount of expression compared to that of tubulin as an internal control using Scion Image software. FIG. 4 (A) describes R-Smad expression. FIG. 4 (B) described I-Smad expression.

FIG. 5 is a set of graphs showing that 1,25-(OH)₂D₃ Treatment Alters MMP and TIMP Expression . FIG. 5 (A) shows that semi-quantitative RT-PCR was used to quantify mRNA levels. S26 was used as the housekeeping gene. FIG. 5 (B) shows protein expression was quantified by ELISA analysis. Fold changes are expressed relative to the allogeneic untreated control.

FIG. 6 is a set of micrographics showing that 1,25-(OH)₂D₃ Treatment Inhibits Glomerular bioactive TGFβ-1 Expression. FIG. 6 (A) shows immunohistochemistry for bioactive TGFβ-1 in a untreated allogeneic graft. FIGS. 6 (B) and (C) show immunohistochemistry for bioactive TGFβ-1 in two 1,25-(OH)₂D₃ treated grafts (1000 ng/rat/day). Tubular staining was observed in all grafts while glomerular staining was significantly decreased in the 1,25-(OH)₂D₃-treated grafts. Magnification 200×.

### DETAILED DESCRIPTION OF THE INVENTION

A. In General

Renal transplantation is the most common form of solid organ transplantation in the United States. Interstingly, patients entering into renal transplantation frequently have aberrant regulation of their vitamin D hormonal axis as a consequence of renal failure. Vitamin D production declines early in the setting of renal insufficiency and vitamin D supplementation is required in many end-state renal disease (ESRD) patients to stabilize parathyroid gland function and calcium status.

Small clinical studies suggest that early after renal transplantation, mild-to-moderate vitamin D deficiency may still exist in up to 40% of patients (P. I. Lobo, et al., Clin. Transplant 9[4]:277-281, 1995; R. Carter, et al., Transplantation 67:S168, 1999). This may be due to abnormalities in vitamin D metabolism inherent in the new allograft, e.g., depressed renal transplant function and unrecognized renal epithelial cell damage. These are manifested by elevated serum creatinine values. (P. I. Lobo, et al., supra, 1995; R. Carter, et al., supra, 1995) The consequences of even relative vitamin D deficiency in this setting have not been examined extensively in the transplant population.

Another aspect of vitamin D activity that may be significantly affected by aberrant vitamin D production and metabolism is the potential immunosuppressive effects associated with vitamin D. Skin (P. Veyron, et al., Transplant Immunol. 1:72-76, 1993), hear (J. M. Lemire, et al., Transplantation 54:762-763, 1992), and kidney transplant (E. Lewin and K. Olgaard, Calcif. Tissue Int. 54:150-154, 1994; D. A. Hullett, et al., Transplantation 66(7):824-828, 1998) survival have all been prolonged by the administration of various vitamin D compounds.

B. Investigation of Calcitriol as a Transplant Therapy

We were interested in determining whether vitamin D compounds, preferably the most common vitamin D supplement 1,25-dihydroxyvitamin D₃ (calcitriol), exerted beneficial effect on renal transplant function. To this end we examined all patients who received calcitriol following kidney or kidney-pancreas transplantation at the University of Wisconsin to determine whether the administration of calcitriol was associated with a change in transplant function. The examples below demonstrate that we found that there were no adverse events identified in association with vitamin D compound therapy and that vitamin D therapy appears to be beneficial in preserving renal graft function in the setting of kidney or kidney-pancreas transplantation. The introduction of the vitamin D compound into a transplant course with declining renal function was associated with stabilization of and preservation of renal function along with a significant deceleration in the rate of loss of function. Calcitriol used early in the post-transplant setting, during the treatment period with the largest dosages of calcineurin inhibitors, was also associated with stable renal graft function without any noted loss of function.

In one embodiment, the present invention is treatment of a kidney transplant recipient with an effective amount of a vitamin D compound, wherein the patient is also treated with immunosuppressant. By “kidney transplant” patient, we mean to include all patients who have had kidney transplants or kidney pancreas transplants.

In a particularly advantageous form of the reaction, the administered compound is either 1α,25-dihydroxyvitamin D₃ (1,25-(OH)₂D₃), 19-nor-1,25-dihydroxyvitamin D₂ (19-nor-1,25-(OH)₂D₃), 24-homo-22-dehydro-22E-1α,25-dihydroxyvitamin D₃ (24-homo-22-dehydro-22E-1,25-(OH)₂D₃), 1,25-dihydroxy-24(E)-dehydro-24-homo-vitamin D₃ (1,25-(OH)₂-24-homo D₃), or 19-nor-1,25-dihydroxy-21-epi-vitamin D₃ (19-nor-1,25-(OH)₂-21-epi-D₃).

In another form of the present invention, the vitamin D compound has the formula 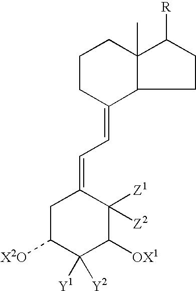
wherein X¹ and X² are each selected from the group consisting of hydrogen and acyl; wherein Y¹ and Y² can be H, or one can be 0-aryl, 0-alkyl, aryl, alkyl of 1-4 carbons, taken together to form an alkene having the structure of 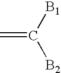
wherein B₁ and B₂ can be selected from the group consisting of H, alkyl of 1-4 carbons and aryl, and can have a β or α configuration: Z¹=Z²=H or Z¹ and Z² together are ═CH₂; and wherein R is an alkyl, hydroxyalkyl or fluoroalkyl group, or R may represent the following side chain: 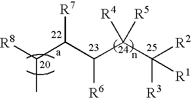
wherein (a) may have an S or R configuration. R¹ represents hydrogen, hydroxy or O-acyl, R² and R³ are each selected from the group consisting of alkyl, hydroxyalkyl and fluoralkyl, or, when taken together represents the group-(CH₂)ₘ-wherein m is an integer having a value of from 2 to 5, R⁴ is selected from the group consisting of hydrogen, hydroxy, fluorine, O-acyl, alkyl, hydroxyalkyl and fluoralkyl, wherein if R⁵ is hydroxyl or fluoro, R⁴ must be hydrogen or alkyl, R⁵ is selected from the group consisting of hydrogen, hydroxy, fluorine, alkyl, hydroxyalkyl and fluoroalkyl, or R⁴ and R⁵ taken together represent double-bonded oxygen, R⁶ and R⁷ taken together form a carbon-carbon double bond, R⁸ may be H or CH₃, and wherein n is an integer having a value of from 1 to 5, and wherein the carbon at any one of positions 20, 22, or 23 in the side chain may be replaced by an O, S, or N atom.

One may evaluate a candidate vitamin D compound for its suitability for the present invention. The candidate compound may first be subjected to an initial rodent-model screening procedure. A successful compound will result in stabilized kidney function or a deceleration in kidney function lost, preferably to the extent shown in the Examples for 1,25-(OH)₂D₃. However, a successful compound is generally described as one that stabilized a patient's serum creatinine levels. The patient should show a significant stabilization of serum creatinine, preferably wherein the level is <3.0 mg/dl, most preferably <2.6 mg/dl, for at least 500 days after transplant.

In another version of the present invention, the rise in serum creatinine levels (indicative of declining renal function) will be reduced compared to patients not treated with vitamin D compound. In one preferred embodiment, the patient's serum creatinine level will not rise more than 1.5 mg/dl compared to level prior to transplant for a period of 500 days after transplant. In another preferred embodiment, the level will not double within 500 days after transplant.

A preferred dose of vitamin D compound for the present invention is the maximum that a patient can tolerate and not develop hypercalcemia. If the vitamin D compound is calcitriol a particularly advantageous daily dose of the compound is between 0.05 and 0.75 μg per day per 160 pound patient. In general, the preferred dose of vitamin D compound is between 0.1 and 50 μg per day per 160 pound patient.

1,25-dihydroxyvitamin D₃ (1,25-(OH)₂D₃) is currently administered at a level of 0.5 μg/day per 160 pound patient, usually in two quarter microgram capsules morning and night, for the treatment of osteoporosis or renal osteodystrophy.

Therefore, the preferred dose of 1,25-(OH)₂D₃ would appear to be at 0.5-0.75 μg/day. Other less active 1α-hydroxy vitamin D compounds can be given at higher doses safely. For example, in Japan the treatment of osteoporosis with 1,25-(OH)₂D₃ is 0.05 to 1.0 μg//day. The same is true of other countries, such as Italy, where as much as 10 μg/day of 1,25-(OH)₂D₃ has been successfully used by Dr. Caniggia (A. Caniggia, et al., Metabolism 39:43-49, 1990).

A preferred mode of treatment is daily, oral administration, preferably with a slow release formulation or a slow release compound. The dose is preferably oral, but could be administered in other manners, such as by injection. Applicants specifically envision that a fairly continuous dosing of vitamin D compound is advantageous in reduction of SLE disease symptoms.

The Examples below describe preferable vitamin D compound dosages ranging between 0.5 μg weekly to 0.75 μg daily.

One would preferably evaluate renal function by assessing serum creatinine values, preferably as described below in Examples. The Examples below disclose that the mean serum creatinine levels increase in patients with no vitamin D compound treatment, thus indicating declining renal function. The data indicate that once calcitriol therapy was initiated, the serum creatinine level stabilized. We define a “stabilized” level as a level <3.0 mg/dl for a period of 500 days after transplant. A “deceleration in kidney functions loss” is defined as slowing the rate of loss of renal function as reflected by no change or minimal fluctuation in serum creatinine values or in other measures of renal function such as creatinine clearance or glomerular filtration rate. We expect a serum creatinine level change of less than 1.5 mg/dl/500 days after transplant.

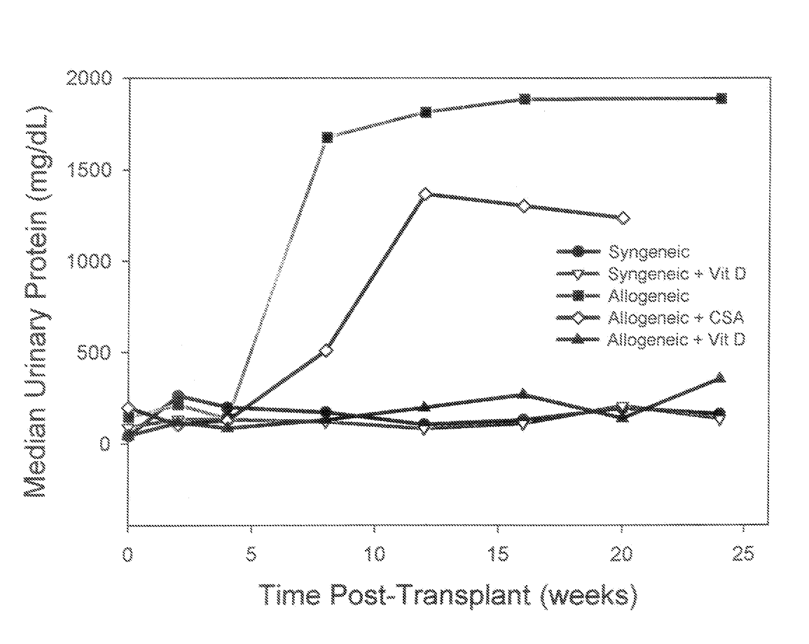

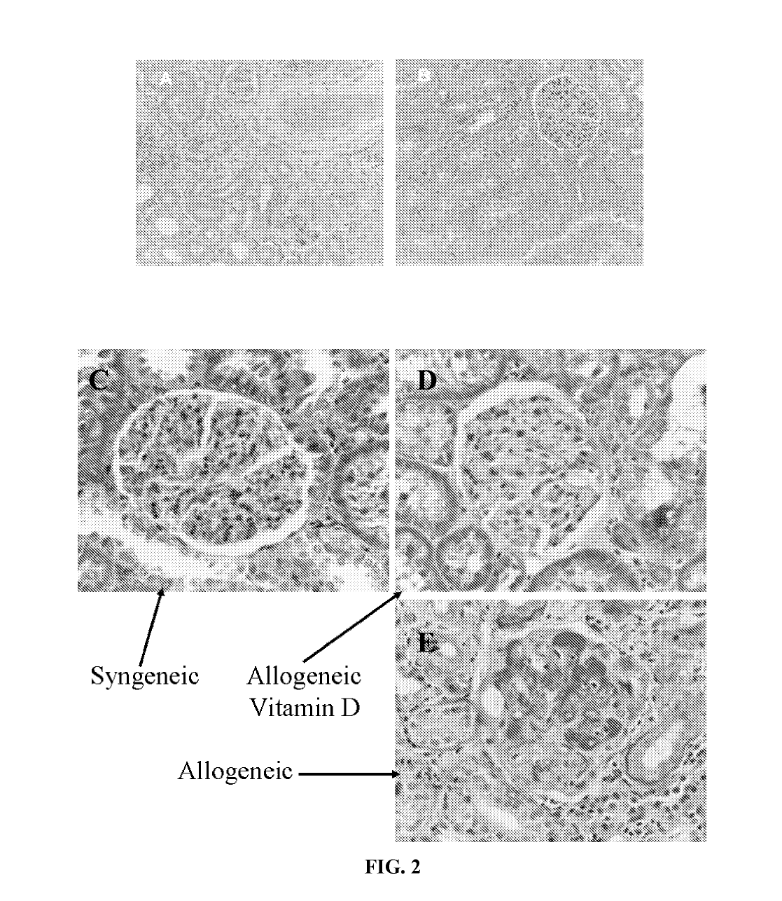

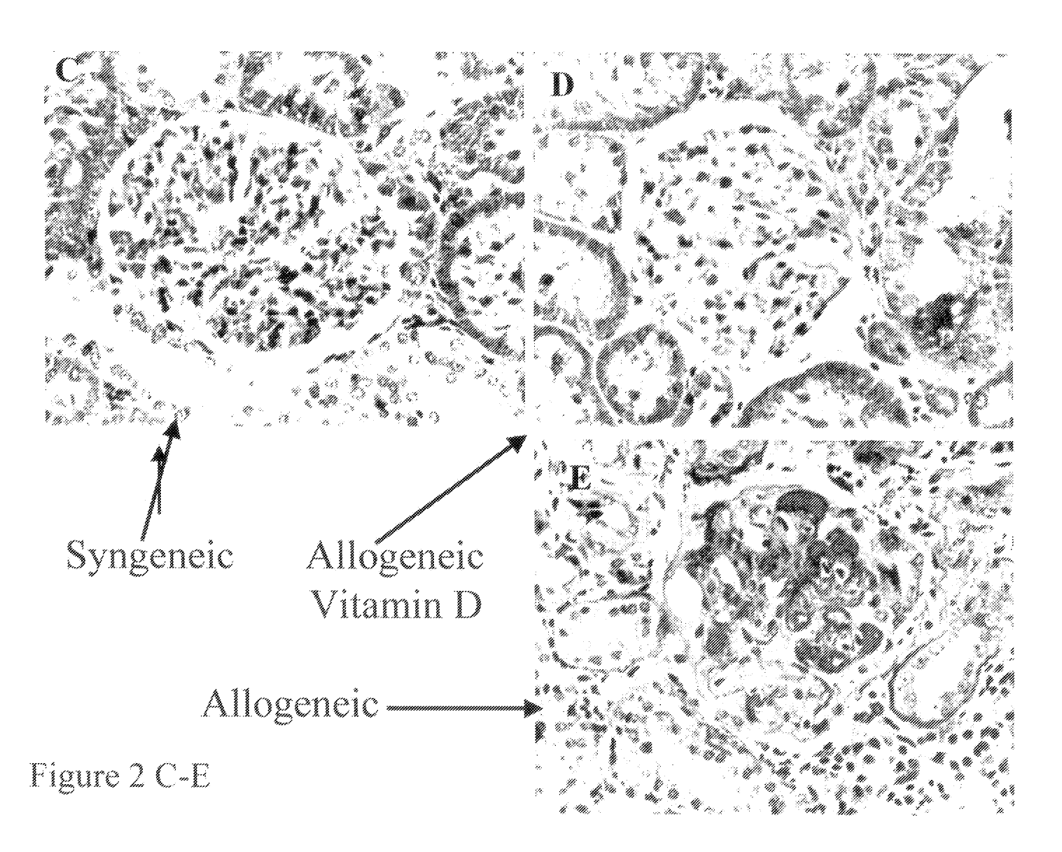

One would also wish to evaluate the effect of treatment by examining the level of fibrotic change throughout the graft. Most notably, the degree of interstitial fibrosis should decrease or remain unchanged with successful treatment. Preferably, one would examine interstitial fibrosis as described below in the Examples, most notably at FIG. 2.

Vitamin D therapy would preferably begin immediately after transplantation or at some point further along in the clinical course after transplantation.

The preferred patient of the present invention had a kidney or kidney-pancreas transplant and has received standard transplant rejection therapies, such as administration of cyclosporine A. Typical immunosuppressive therapy would include: corticosteroids, cyclosporine A or tacolimus, mycophenolate mofetil and/or use of rapamycin or rapamycin analogs and/or azathioprine.

### EXAMPLES

### I. Effects of 1,25(OH)₂D₃ on Renal Transplant Patients

We examined all patients who received calcitriol following kidney or kidney-pancreas transplantation at the University of Wisconsin to determine whether the administration of calcitriol was associated with a change in transplant function.

Methodology-retrospective analysis: The University of Wisconsin transplant database was screened to identify any kidney and/or kidney-pancreas transplant recipient who received 1,25-dihydroxyvitamin D₃ (calcitriol)peri-post-transplant. Clinical and demographic variables were abstracted from the database. Those patients with adequate follow-up data were included in the analysis (≧1year follow-up data following the initiation of calcitriol). Demographic variables included race, age, and nay history of parathyroidectomy as these patients would likely be receiving calcitriol at time of transplantation. The effect of calcitriol treatment on renal function was analyzed using general linear mixed modeling of the change in slope of renal function prior to and following the start of calcitriol therapy. The effect of calcitriol on cyclosporine A (CsA) and tacrolimus (FK506) serum levels was analyzed by standardizing the milligram dosages of these agents to a mean of 0 with a standard deviation of 1. Adverse events and hypercalcemia were noted by identifiers in the database and by serum calcium levels as recorded in the database. Hypercalcemia was defined as a serum calcium >10.5 mg/dl.

Data assessment—Demographics: Calcitriol-treatment patients were divided into:

Group 1: patients who initiated calcitriol therapy<one year following transplantation,

Group 2: patients who remained on or initiated calcitriol therapy within two weeks of transplantation,

(A third group of patients were also identified. These patients were started on calcitriol therapy between 15 days and 1 year following transplantation. These patients have not been completely evaluated at this point in time.)

The demographic characteristics for Group 1 and Group 2 patients are shown in Table 1. When appropriate, these data are represented as mean±standard deviation. The vast majority of patients in both groups were caucasian.

TABLE 1
Demographics for calcitriol-treated patients

| Characteristic | Group 1 | Group 2 |
|---|---|---|
| No. | 26 | 22 |
| Average age at Tx (yrs) | 41.3 ± 11.8 | 46.5 ± 14.5 |
| M/F | 16/10 | 13/9 |
| Caucasian/African- | 24/1/1 | 18/3/1 |
| American/Other | | |
| Donor age* | 31 ± 16.7 | 28.6 ± 14 |
| Cadaver/live donor Tx** | 15/9 | — |
| Pre-Tx parathyroidectomy | 5 | 3 |

*donor age available for n = 15 in Group 1;
**data available for n = 24 in Group 1, not available for Group 2 patients

Type 1 diabetes mellitus was the most common cause or etiology of end-stage renal disease (ESRD) in Group 1 patients (n=9) (Table 2). A variety of other etiologies accounted for ESRD in the remaining patients. All of these entities also occurred in the Group 2 patients though with a different prevalence (Table 2).

TABLE 2
Etiologies for ESRD in Group 1 and
Group 2 calcitriol-treated patients

| Cause of ESRD | Group 1 | Group 2 |
|---|---|---|
| Type 1 diabetes mellitus | 9 | 2 |
| Chronic glomerulonephritis | 1 | 2 |
| Hypertension | 3 | 2 |
| Focal segmental | 3 | 1 |
| glomerulosclerosis | | |
| IgA nephropathy | 1 | 2 |
| Membranous glomerulonephritis | 3 | 1 |
| Other | 6 | 12 |

Calcitriol dosages ranged between 0.5 μg weekly to 0.75 μg daily, with no significant different between dosage ranges between Group 1 and Group 2.

Data assessment—outcomes for Group 1 patients: Renal function was determined by assessing serial serum creatinine values. This serum measure is a standard and accepted measure of renal function. The start of calcitriol therapy was defined as day 0. Time prior to initiation of calcitriol therapy was designated by a negative value. Mean serum creatinine levels appeared to increase in Group 1 patients (indicative of declining renal function) until the time of calcitriol therapy was initiated (Table 3).

TABLE 3
Serum creatinine values in Group 1 patients prior
to and following initiation of calcitriol therapy.

| Day | Mean serum creatinine (mg/dl) | Standard deviation |
|---|---|---|
| −500 | 1.39 | 0.52 |
| −400 | 1.52 | 0.48 |
| −300 | 1.68 | 1.23 |
| −200 | 1.91 | 1.09 |
| −100 | 2.65 | 1.44 |
| Therapy | | |
| Administered | | |
| 100 (calcitriol) | 2.54 | 2.26 |
| 200 (calcitriol) | 2.44 | 1.26 |
| 300 (calcitriol) | 2.50 | 1.21 |
| 400 (calcitriol) | 2.27 | 1.34 |
| 500 (calcitriol) | 2.37 | 1.47 |

Serum creatinine levels stabilized following initiation of calcitriol therapy. These results were substantiated by a repeated measures analysis of variance of the slopes of creatinine trends over time. This analysis indicated that the rate of increase in serum creatinine was greatest in the interval immediately pre-calcitriol therapy (0.007 mg/day) (p=0.009 vs. calcitriol therapy period). After 300 days of calcitriol therapy, creatinine was decreasing evidenced by a negative slope, suggesting a significant stabilization or deceleration of the rate of loss of renal graft function with therapy. This is demonstrated in Table 4 in which “difference” is defined as the change in slope of a patient's serum creatinine plotted over time. A negative value for this slope denoted a decreasing slope and improved renal function.

TABLE 4
Change in slope of creatinine over time in Group 1 patients.

| Treatment day | Difference | P value |
|---|---|---|
| +100 | −0.0050 | 0.128 |
| +200 | −0.0046 | 0.187 |
| +300 | −0.0080 | 0.031 |
| +400 | −0.0085 | 0.031 |
| +500 | −0.0078 | 0.041 |

Ultimately, six patients in this group had graft failures (loss of the transplant).

Data assessment—outcomes for Group 2 patients: The start of calcitriol therapy was again defined as day 0. Mean serum creatinine levels remained stable in this patient cohort for the first 600 days following initiation of calcitriol (Table 5).

TABLE 5
Serum creatinine values in Group 1 patients prior
to and following initiation of calcitriol therapy

| | Mean serum | |
|---|---|---|
| Day | creatinine (mg/dl) | Standard deviation |
| 100 (calcitriol) | 1.61 | 0.65 |
| 200 (calcitriol) | 1.79 | 0.50 |
| 300 (calcitriol) | 1.70 | 0.37 |
| 400 (calcitriol) | 1.62 | 0.44 |
| 500 (calcitriol) | 1.82 | 0.49 |

The analysis of slopes found no interview slopes significantly different from 0 nor any interval slopes significantly different from one another. These data suggested that calcitriol therapy was associated with stabilization of early renal graft function in the setting of kidney and kidney-pancreas transplantation.

Ultimately, there were two graft failures in this group.

Data assessment—effect of calcitriol on immunosuppressive agents: The calcineurin inhibitors, cyclosporine A (CsA) and tacrolimus (FK506) are standard immunosuppressive agents for kidney and kidney-pancreas transplant recipients. Both of these agents have beneficial effects in prolonging allograft function by altering the ability for activated T cells to produce interleukin-2 (IL-2). However, both of these agents are also associated with drug-related nephrotoxicity that is manifested by characteristic histologic changes in the allograft, e.g. tubulointerstitial fibrosis, and loss of allograft function long-term. Thus, it was important to note in this retrospective analysis whether calcitriol altered CsA or FK506 serum levels, potentially altering their immunosuppressive effects on the immune system and their potential long-term nephrotoxic effects.

Seven of the 26 Group 1 patients had serial CsA levels following the initiation of calcitriol therapy. No trends in either mean CsA levels or variability in CsA levels were noted.

To make the calcineurin inhibitors relatively comparable for the purposes of statistical analyses, the milligram dosages for each were standardized to a mean of 0 and a standard deviation of 1. All of the Group 2 patients were treated either with CsA or FK506 therefore they were analyzed in combination with the Group 1 CsA-treated patients to determine the effect of calcitriol, if any, on drug dosing requirements. Calcitriol therapy had no significant effect on CsA or FK506 dosages or serum drug levels in a combined analysis of all Group 1 and Group 2 patients and there were no trends influenced by calcitriol in CsA of FK506 dosage requirements or serum drug levels.

Data assessment—calcitriol and adverse events: There were no adverse events identified in association with calcitriol therapy. The mean serum calcium in Group 1 and Group 2 patients was <10 mg/dl (9.6±0.9 mg/dl). There were no episodes of sustained hypercalcemia (>2 serial hypercalcemia values noted) or hypercalcemia events requiring hospitalization. There were no episodes of substained hematuria that could be directly attributable to hypercalcemia.

Summary—calcitriol therapy in kidney and kidney-pancreas transplantation: Calcitriol therapy appears to be beneficial in preserving renal graft function in the setting the kidney or kidney-pancreas transplantation as determined in this retrospective study. The introduction of calcitriol into a transplant course with declining renal function was associated with stabilization of and preservation of renal function along with a significant deceleration in the rate of loss of function. Calcitriol use early in the post-transplant setting, during the treatment period with the largest dosages of calcineurin inhibitors, also was associated with stable renal graft function without any noted loss of function.

### II. Effects of 1,25(OH)2D3 on Fisher to Lewis Renal Allograft Model

Chronic allograft nephropathy (CAN) is characterized by the development of fibrotic changes throughout the allograft including glomerulosclerosis, interstitial fibrosis, tubular atrophy, and concentric neointimal hyperplasia. CAN is irreversible ultimately resulting in patient retransplantation or dialysis. The mechanisms underlying the development of CAN are unknown, but likely involve a complex interaction between humoral and cellular immune responses, cold ischemia/perfusion injury and cytokine expression, particularly TGFβ-1. The role of TGFβ-1 in CAN has recently been reviewed (Jain, et al. Transplant, 2000 69 1759-1766). Notably, several transplant studies have correlated TFGβ-1 expression with the development of interstitial fibrosis and glomerulosclerosis in kidney transplant recipients (Sime et al., J. Clin Invest. 100:768-776, 1997; Cohen & Nast, Min Electrolyte Metab 24:197-201, 1998; Suthanthiran, A,. J. Med. Sci. 313:264-267, 1997; Shihab, et al. Transplantation 64:1829-37, 1998).

It is important to recognize 1,25-(OH)₂D₃'s mechanism of action in attempting to understand its effects in a transplant setting. 1,25-(OH)₂D₃ traverses the cytoplasmic membrane where it binds the vitamin D receptor (VDR). VDR or VDR complexed with retenioic acid receptor (RXR) then travels to the nucleus where it functions in conjunction with other co-activator/repressors as a transcription to differentially affect the expression of various genes, depending on cellular phenotype, cell cycle, and cellular activation Strugnell and DeLuca, Proc. Soc. Exp. Biol. Med. 245:223-228, 1997. There is a direct link between the 1,25-)OH)₂D₃ and TGFβ-1 pathways. TGFβ-1 binding to its cell surface receptor results in the phosphorylation of the receptor-activated Smads 2/3 which then interact with the co-Smad 4 to form a heterodimeric complex which translocates to the nucleus to regulate gene expression. We and others have shown that Smad 3 forms a complex with the VDR, both in vivo and in vitro. Yanagisawa, et al., Science 283: 1317-1321, 1999; Aschenbrenner et al. Transplant 70 S, 2001. This suggests that 1,25-(OH)₂D₃ may regulate TFGβ-1-mediated gene and protein expression and, therefore may alter TGFβ-1 effects in CAN.

Here we examined the effects of 1,25-(OH)₂D₃ therapy in the Fisher to Lewis renal allograft model, a model of CAN. Out results suggest that 1,25-(OH)₂D₃ is effective in prolonging allograft survival and limiting CAN in this model.

Material and Methods

Animals: Donor and recipients rats (greater than 250 gm) were obtained from Harlan Sprague Dawley, Indianapois, Ind. Recipient animals were placed on experimental diet containing 0.47% Ca 7 days prior to transplantation. Hullett et al., Transplantation 66:824-828, 1998. Recipients were divided into groups which received experimental diet alone or experimental diet containing 1,25-(OH)₂D₃ (250, 500, or 1000 ng/rat/day) or cyclosporine 1.5 or 5 mg/kg/day i.p. for 10 days. Animals were maintained on diet until the time or rejection or graft harvest at 24 weeks. All care and use of laboratory animals followed the NIH (NIH publication No. 86-23) guidelines. 1,25-(OH)₂D₃ was prepared, dissolved in ethanol and placed in the experimental diet as previously described.1,25-(OH)₂D₃ was the generous gift or Dr. Hector DeLuca, Department of Biochemistry, University of Wisconsin. Cyclosporine (Sandimmune i.v.; 1.5 or 5 mg/kg/day for 10 days,) was diluted in saline and given i.p.

Transplantation: The Fisher to Lewis model of chronic allograft nephropathy has been previously described. Diamond et al., Transplantation 54:710-716, 1992. Briefly, donor kidneys obtained from male Fisher 344 rats were flushed with 10 ml cold University of Wisconsin preservation solution and stored at 4° C. while the recipient was prepared. Total cold ischemic time did not exceed 30 minutes. Lewis male recipients were transplanted with either a Fisher 344 or Lewis kidney following left native nephrectomy. Briefly, the donor renal artery, vein and ureter were anastomosed to the recipient renal artery, vein and ureter. The right native kidney was removed 10 days post-transplant. Graft function was monitored by serum creatinine and urinary protein determinations.

Urinary Protein: Proteinuria was assessed weekly. Animals were placed in metabolic cages for 6 hours and urine collected. Protein excretion was determined using a dye binding assay (quanTest red, Quantimetrix Corp., Redondo Beach, Calif.) according to manufacturer=s instructions with minor modifications. Briefly, 20 μl of the urine sample was diluted with 1×PBS in a two-fold series dilution in 96-well flat-bottom microtiter plates (Corning, N.Y.). The final dilution was 1:32. 125 μl quanTest red reagent was added and the protein concentration in the samples measured by reading the absorbance at 600 nm and compared to the absorbance of a 50-0.062 mg/ml rat albumin/globulin protein standard on a Vₘₐₓ Kinetic microplate reader (Molecular Devices, Sunnyvale, Calif.). Data was analyzed with Softmax Pro-software (Molecular Devices Corp.; Sunnyvale, Calif.).

Semi-quantitative Reverse transcription polymerace chain reaction (RT-PCR): Following graft harvest a protein containing both cortex and medulla was snap frozen in liquid nitrogen. Semi-quantitative RT-PCR was performed as described. Little et al. Transplant Int. 12:393-401, 1999. Briefly, samples were homogenized then total RNA extracted with RNAzol B (Tel Test, Inc., Friendswood, Tex.) and reversed transcribed to cDNA according to manufacturer=s instructions (Superscript, Gibco BRL). PCR was performed over the linear range of amplification for both the gene of interest, e.g. TGFβ-1, and the ribosomal protein S26 as a control housekeeping gene. PCR conditions were chosen such that both products were amplified with similar efficiency. The following cycle parameters were employed: denaturing, 94° C. for 60 seconds; annealing, 61° C. for 60 seconds; and extension, 72° C. for 60 seconds. The amount of product was quantified using a phosphoimager (Storm 80) following gel electrophoresis and Vista green detection.

Western Blot Analysis: A portion of the snap frozen graft containing both cortex and medulla was ground with a mortar and pestle. For each 100 mg of tissue, the sample was resuspened in 400 ul of lysis buffer (10 mM Tris base, 150 mM NaCl, pH 8.0) containing protease inhibitors (P2714 1:1000. Sigma Chemical Co., St. Louis, Mo.) and homogenized (PowerGen 125, 1 minute). Triton X100 was then added to 1% and the sample place on ice, for 30 minutes. The solubilized sample was then spun at 4° C. for 15 minutes at 15,000×G. The supernatant was collected and stored at −80° C. Sample protein concentrations were determined using the Micro BCA assay (Pierce Chemical Co., Rockford, Ill.) according to manufacturer=s instructions, except that the assay was performed in half area plates combining 35 μl sample with 70 82 l of reagent. Sample (30 μg/well) were resolved on a 10% reducing acrylamide gel and transferred to a nitrocellulose membrane using a semi-dry transfer system (Bio-Rad Laboratories, Hercules, Calif.). Membranes were blocked overnight at 4° C. in Blotto B (1% dry milk, 1% BSA, 0.05% tween 20 in PBS) plus 0.05% sodium azide. Membranes were washed with PBS tween (6×-10 minutes) followed by addition of diluted primary antibody (Blotto B +5% milk, 1 hour at RT with rocking; rabbit anti-Smad 2, 3, 6, Zymed Corp., San Francisco, Calif. 1:6000; goat anti-Smad 7, Santa Curz, Santa Cruz Calif., 1:2500; mouse anti-tubulin clone Ab-4, Neomarkers, Fremont Calif., 1:14,000). Membranes were again washed and diluted secondary HRP-conjugated antibody added (anti-rabbit IgG-HRP 1:32,000, Transduction Labs, Lexington, Ky.; anti-goat IgG-HRP 1:2500, Transduction Labs; anti-mouse IgG-HRP 1:240,000, Transduction Labs). The membranes were washed and (four 10-minutes washes with PBS-tween followed by two 10-minutes PBS washes) and then developed using the SuperSignal West Pico Chemiluminescent substrate according to manufacture=s instruction (Pierce Chemical Co.)

Enzyme Linked Immunoabsorbant Assay (ELISA): TGFβ-1 and MMP 2 levels were quantified by antigen-capture ELISA. A flat bottomed, half area EIA/RIA A/2 plate (Costar, Cambridge, Mass. USA) was coated overnight at 4° C. with 25 μL monoclonal primary antibody (anti-TGFβ-1, IgG 1:1000 dilution in carbonate buffer, pH 9.7, TGFβ-1 Eₘₐₓ ImmunoAssay, Promega Inc., Madison Wis.; anti-MMP2, 2.5 ug/ml, clone 1A10, R and D Systems, Minneapolis, Minn.). After blocking with 1× block buffer at 37° C. for 35 minutes 25 ul of cell lysate (diluted 1:10 in the lysis buffer) was added to the wells. A standard curve was generated by performing two fold serial dilutions of the standard active TGFβ-1 antigen (diluted 15.6 pg/ml to 1000 pg/ml, Promega, Inc.) or MMP2 (0.5 ng/ml to 100 ng/ml, R and D Systems). The plate was incubated for 2 hours at RT with shaking and then washed extensively with wash buffer (0.05% Tween 20 in PBS) followed by PBS. The TGFβ-1 ELISA was then developed according to the manufacturer's instruction. For the MMP2 ELISA, biotinylated detection antibody, clone 101721 (25 μL/well at 1.4 ug/ml R and D Systems) was added and incubated at RT (1hour, with shaking). The antibody was biotinylated using a Mini-Biotin-XX Protein Labeling Kit (F-6347) according to manufacturer's instruction (Molecular Probes, Eugene Oreg.). Following further washing, avidin-peroxidase conjugate was added (25 ul/well at 1:5000) for 30 minutes at RT. A color reaction was developed by the addition of 25 ul of the TMB (3, 3′, 5, 5′-tetramethylbenzidine)/hydrogen peroxidase substrate solution, (KPL, Gaithersberg, Md.). Color development was stopped after approximately 10 minutes by the addition of 25 ul TMB stop solution. Absorbance was measured at 450 nm on a Vₘₐₓ Kinetic microplate reader (Molecular Devices, Sunnyvale Calif.). To measure total TGFβ-1 in the sample, acid activation was performed: 1 ul of 1N HCl was added to the harvested supernatant sample (diluted 1:5 in PBS) and incubated at RT for 15 minutes. One ul 1M NaOH was then added to neutralized the acid. Acid activated samples were then assayed by antigen-capture ELISA after a further 1:10 dilution in sample buffer.

Immunhistochemistry: Formalin fixed, paraffin embedded rat kidneys were sectioned to 4 microns. Sections were rewarmed on day of staining at 60° for 10 minutes, then deparaffinized in xylene for 30 minutes followed by rehydration. Sections were washed twice in distilled water and antigen retrieval performed. Briefly, section were soaked in 100 mM citrate buffer, pH=6.0 for 10 minutes at 90° C. and then heated to 115° C. or 20 minutes Sections were allowed to cool and then washed in PBS and blocked for 10 minutes with BIOCARE SUPER SNIPER9 (BS996L) followed by diluted primary TGFβ-1 antibody (1:150 (Promega Corp., Madison, Wis.; G1221) at 4° C. overnight. Slides were washed (3×5 minutes, PBS) and secondary antibody applied (1 hour at RT, MACHII BIOCARE goat anti-rabbit with polymer spacer; RHRP52OH, Walnut Creek, Calif.).

Slides were developed with Pierce=s Stable Peroxide buffer (cat #1855910) and Pierce=s Stable Metal enhanced DAB solution (cat #1856090).

Statistical Methods:

Graft survival was compared using the log rank test. Differences in mRNA and protein expression were compared by t-test.

Results

1,25-(OH)₂D₃ Prolongs Allograft Survival and Decreases the Severity of CAN.

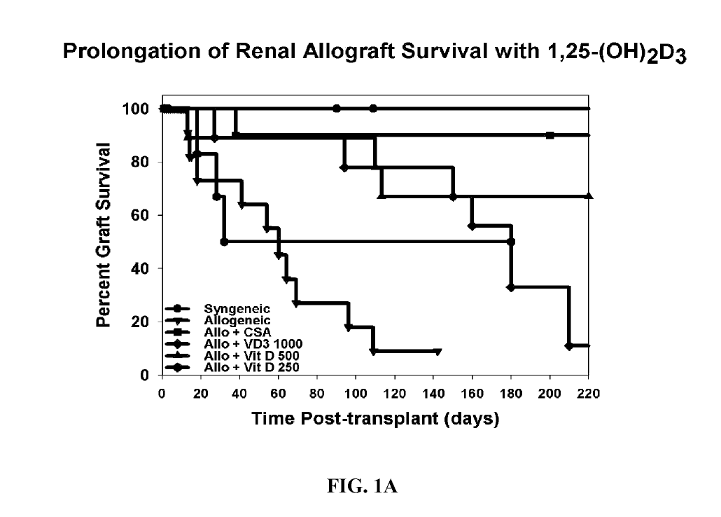

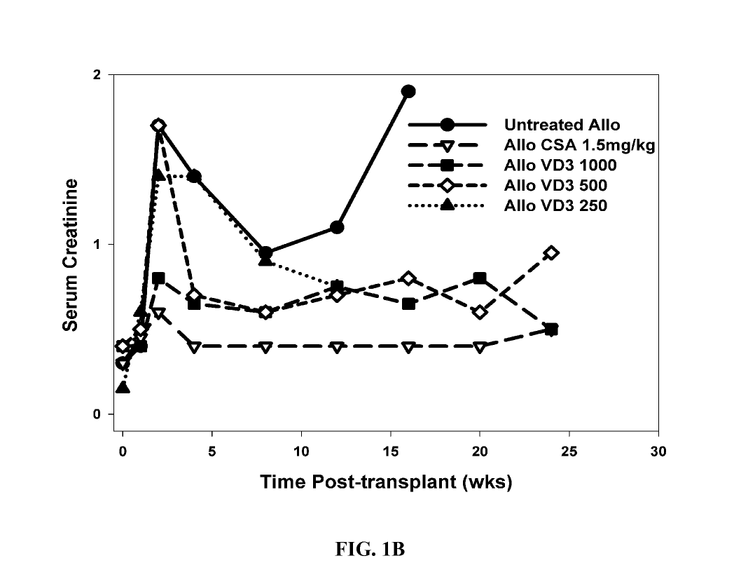

Dietary 1,25-(OH)₂D₃ (1000 ng/rat/day, monotherapy) significantly prolonged graft survival in allogeneic recipients (FIG. 1, p=0.0031) in comparison to allogeneic untreated controls. When 1,25-(OH)₂D₃ was reduced to 500 ng/rat/day significant prolongation of graft survival was sustained (p=0.0009), but at 250 ng/day prolonged graft survival was not as readily demonstrated (p=0.04). Prolonged graft survival at 1000 or 500 ng/rat/day was not statistically different from recipients treated with low dose CSA (5 mg/kg/day for 10 days).

In this model, an early acute rejection (within 2 weeks post-transplant) episode typically occurs that is prevented with short-term low dose CSA monotherapy (FIG. 1B). Treatment with 1,25-(OH)₂D₃ also diminished the early acute rejection episode. This resulted in only a slight increase in serum creatinine at 2 weeks post-transplant (FIG. 1B, p=0.035 versus untreated allogeneic control). Neither the 500 or the 250 ng/day dose of 1,25-(OH)₂D₃ prevented the rise in serum creatinine at 2 weeks post-transplant (FIG. 1b).

We also determined urinary protein excretion following transplantation. As shown in FIG. 1C, monotherapy with 1,25-(OH)₂D₃ 1000 ng/rat/day (p=0.004) or 500 ng/rat/day (data not shown) significantly lowered urinary protein in comparison to untreated allogeneic controls or allogeneic recipient treated with 1.5 mg/kg/d (10 days) CSA.

Histological examination of the untreated allografts demonstrated features characteristic of CAN including interstitial fibrosis, glomerulosclerosis and neointimal hyperplasia (FIG. 2A). In contrast 1,25-(OH)₂D₃ treatment inhibited the development of these pathological features (FIG. 2B). When sections were stained with trichrome to visualize collagen deposition, analysis revealed decreased collagen deposition in 1,25-(OH)₂D₃-treated recipients and preservation of glomerular structure (FIG. 2D-E).

Significant calcium deposits were observed in recipients treated with 1000 ng 1,25-(OH)₂D₃. While serum calcium levels remained elevated in both the 250 and 500 ng/day groups (data not shown) histological examination showed reduced calcium deposition with evidence of a mild to moderate cellular infiltration (Banff IA or IIA).

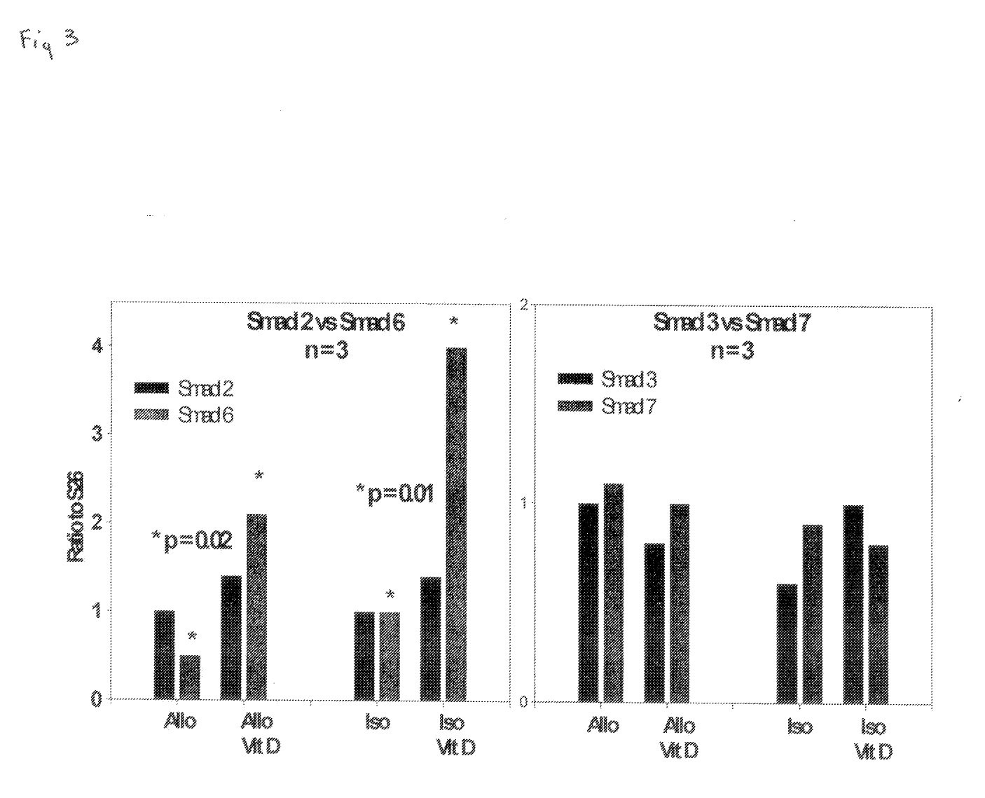

1,25-(OH)₂D₃ Treatment Altered Allograft Smad Expression. Semi-quantitative RT-PCR analysis showed no significant change in the expression of Smad 2, Smad 3 or Smad 7 mRNA levels. However, Smad 6 mRNA expression was significantly increased in both allogeneic (p=0.02) and syngeneic (p=0.001) grafts treated with 1,25-(OH)₂D₃ (FIG. 3) in comparison to untreated allogeneic grafts. No change in Smad 2, 3, 6, or 7 mRNA levels was observed in the CSA treated recipients in comparison to either allogeneic or syngeneic controls. Smad 6 mRNA expression was significantly elevated (p=0.048) in comparison to the untreated allogeneic control group and was not significantly different from the untreated allogeneic control group and was not significantly different from the untreated syngeneic control group (p=0.27). Vitamin D receptor mRNA expression was elevated in all groups receiving 1,25-(OH)₂D₃ therapy in comparison to the untreated syngeneic control, but was not significantly different from the amount of expression in the untreated allogeneic control (data not shown).

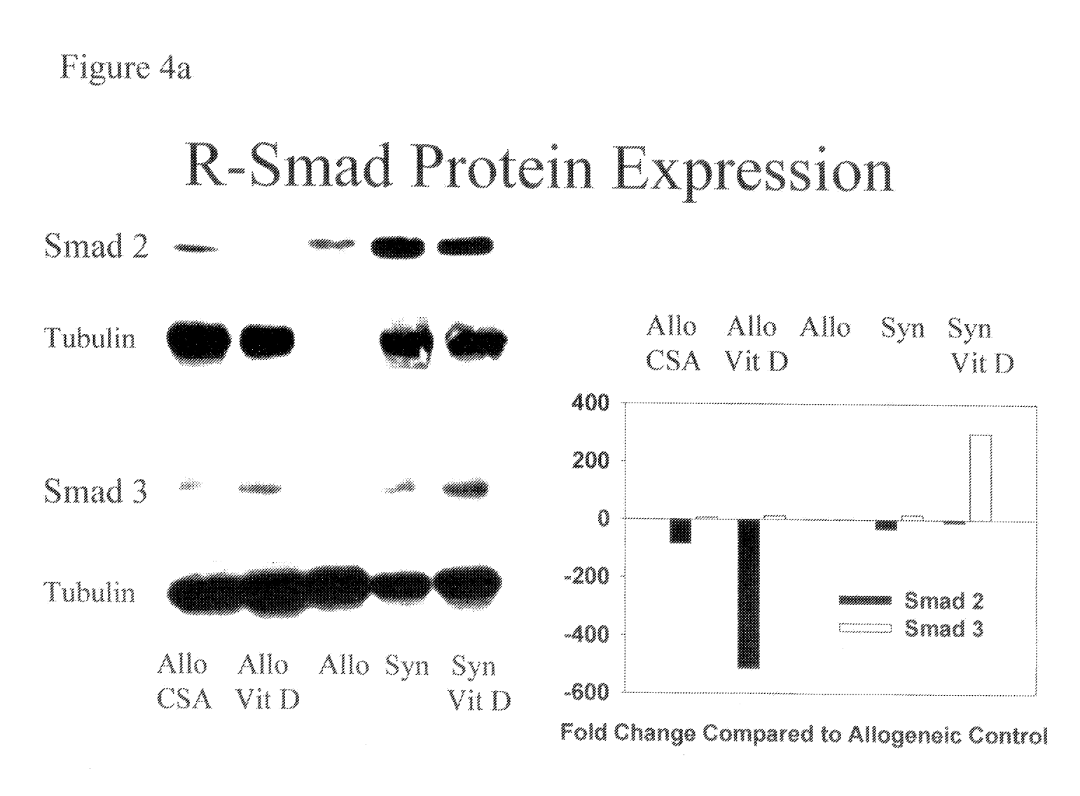

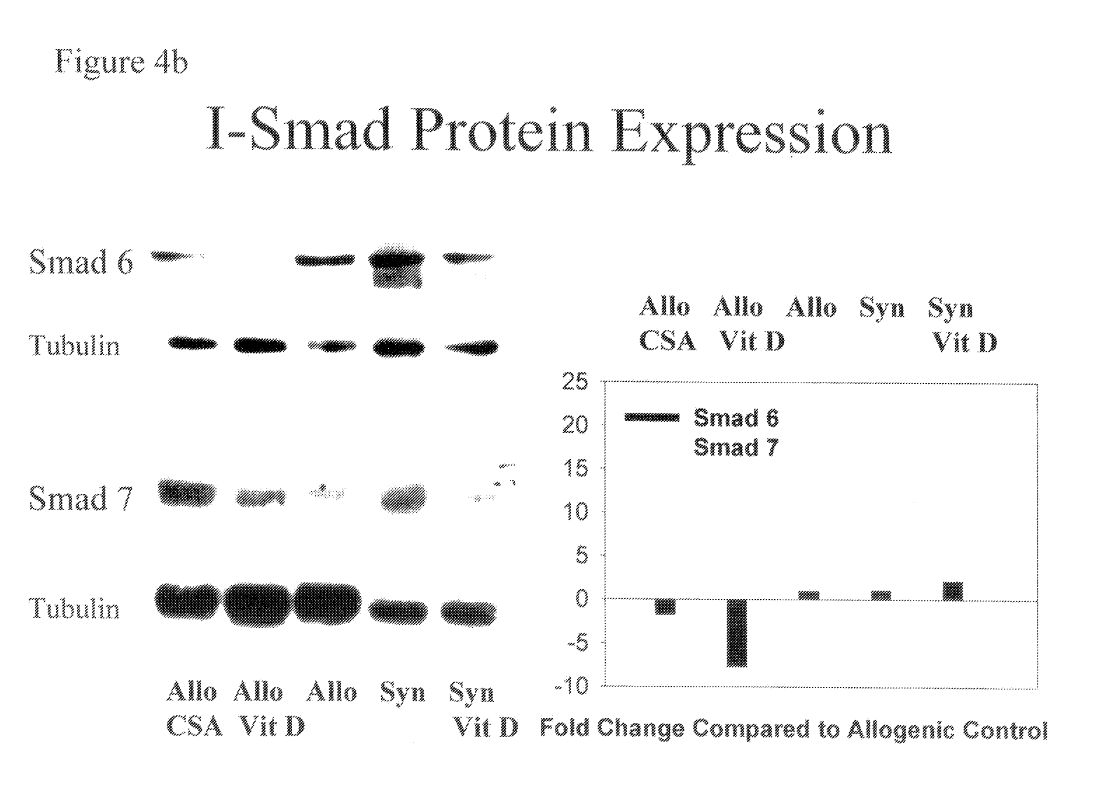

Immunoblotting revealed a dramatic decrease in Smad 2 (FIG. 4A, p=0.04) protein levels in comparison to allogeneic untreated controls in recipients treated with either 1000 (516-fold) or 500 (208-fold, data not shown) ng/rat/day. Smad 3 protein levels were similar in allogeneic recipients regardless of treatment in comparison to syngeneic controls (data not shown). Smad 7 protein levels were increased in both 1,25-(OH)₂D₃-treated (4,3-fold, p=0.02) and CSA-treated (5.4-fold p=0.03) allogeneic grafts in comparison to untreated allogeneic grafts (FIG. 4B). However, Smad 7 expression in allogeneic recipients was decreased in comparison to syngeneic controls. Smad 6 protein expression was decreased in both the 1,25-(OH)₂D₃ and CSA treated allograft recipients and was unchanged in syngeneic recipients in comparison to the allogeneic untreated control group.

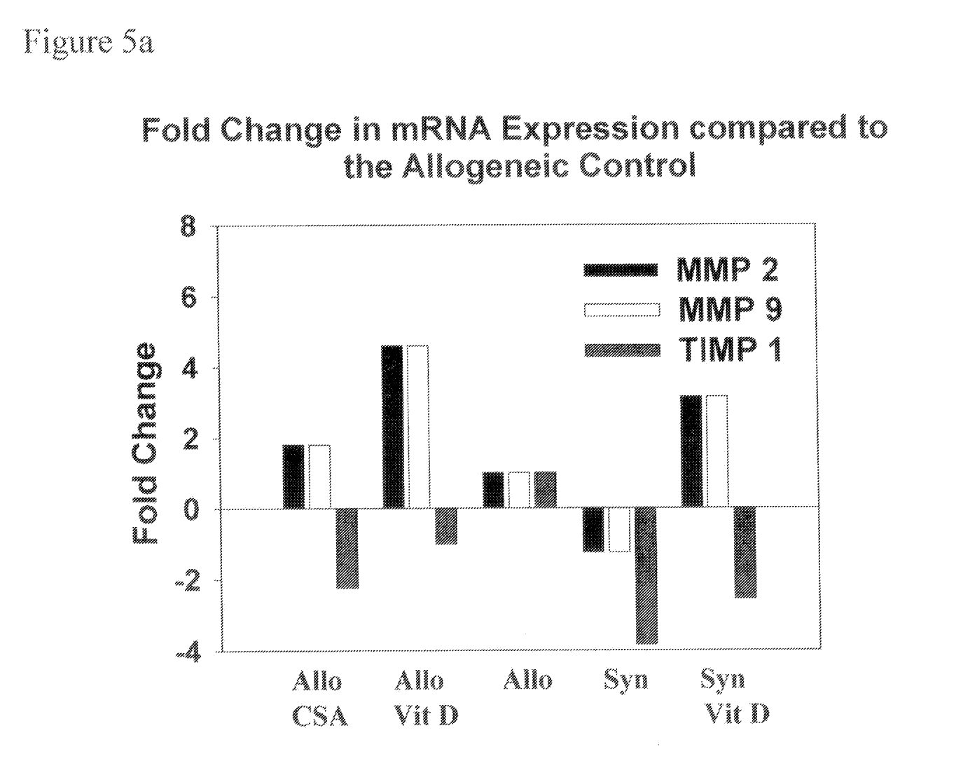

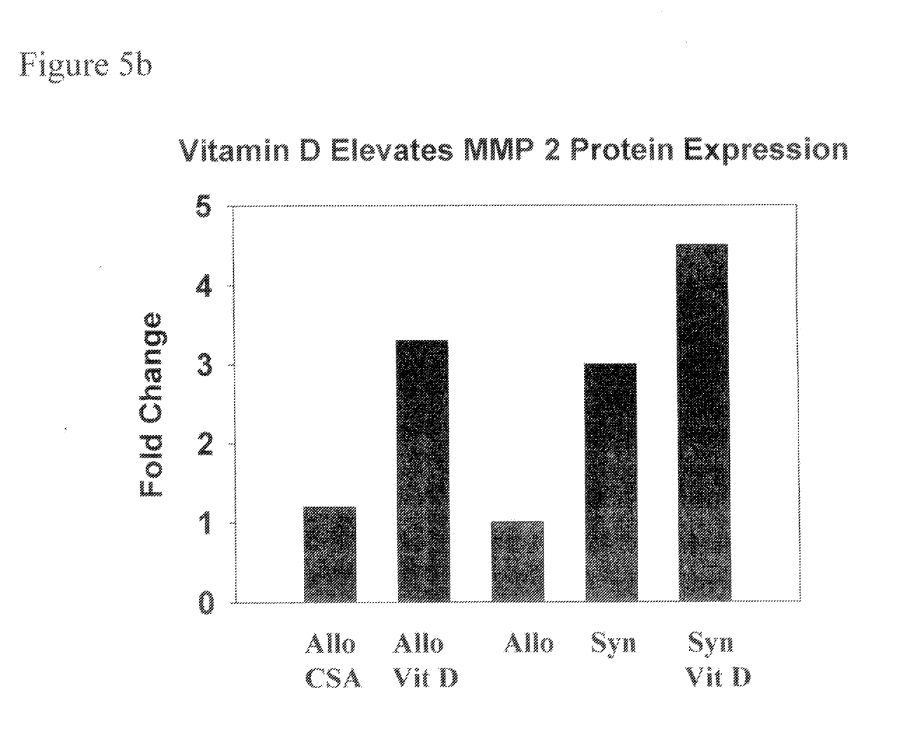

1,25-(OH)₂D₃ Altered Allograft MMP and TIMP Expression: Semi-quantitative RT-PCR analysis was performed to examine grafts for changes in MMP and TIMP mRNA levels. As shown in FIG. 5A, 1,25-(OH)₂D₃ treatment significantly increased MMP 2 mRNA levels in both allo-(5.6-fold) and syngeneic (4.0-fold) grafts. MMP 9 mRNA was increased in all groups in comparison to syngeneic untreated controls regardless of 1,25-(OH)₂D₃ treatment. TIMP 1 mRNA expression was decreased in all groups in comparison to the untreated allograft recipients regardless of treatment. Total MMP 2 protein expression was increased in both the syngeneic and allogeneic 1,25-(OH)₂D₃-treated groups and was unchanged in the CSA-treated allogeneic recipients in comparison to the untreated allogeneic group (FIG. 5B).

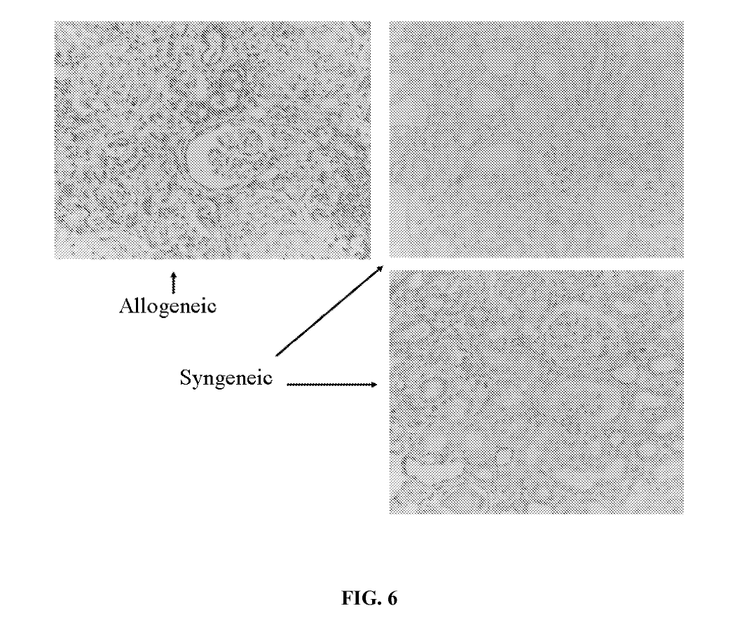

1,25-(OH)₂D₃ Treatment Does Not alter TGFβ-1 mRNA or Protein Expression: Semi-quantitative RT-PCR and antigen-capture ELISA for bioactive TGFβ-1 analysis revealed no significant difference changed in TGFβ-1 mRNA or protein levels in the 1,25-(OH)₂D₃-treated recipients in comparison to untreated allogeneic control grafts. Interstingly, both TGFβ-1 mRNA (2.3-fold) and bioactive protein (2.3-fold) expression were elevated in the CSA-treated allogeneic recipients. As shown in FIG. 6, immunohistochemistry for bioactive TGFβ-1 revealed extensive staining in tubular areas of 1,25-(OH)₂D₃-treated and non-treated allogeneic grafts. However, little bioactive TFGβ-1 expression was observed in the glomeruli of 1,25-(OH)₂D₃-treated grafts in contrast to the untreated control grafts.

Discussion

The results described herein demonstrate the efficacy of 1,25-(OH)₂D₃ in this model of CAN. 1,25-(OH)₂D₃ significantly prolonged allograft survival. In addition 1,25-(OH)₂D₃ therapy stabilized or prevented histologic changes associated with CAN. The data also suggest that 1,25-(OH)₂D₃ therapy altered the expression of signaling molecules integral to TGFβ-1-regulated gene expression, affecting gene/protein expression likely to have important roles in CAN. We observed a dramatic reduction in Smad 2 protein expression with concomitant increase in Smad 7. We also observed significant changes in genes regulated by TGFβ-1; MMP 2 mRNA and protein expression were increased while TIMP 1 gene expression was decreased. Our data clearly suggest that 1,25-(OH)₂D₃ therapy improved allograft function in conjunction with changes in molecules directly related to ECM remodeling. This may be a unique kidney-specific role of 1,25-(OH)₂D₃ in addition to effects 1,25-(OH)₂D₃ may have on immune responses.

Most studies have shown marginal prolongation of graft survival by 1,25-(OH)₂D₃ (Bouillon, et al., Endocrine Review 16:200, 1995). In all cases, significant hypercalcemia was observed. With one exception, 1,25-(OH)₂D₃ in these studies was administration by daily or alternate day i.p. injection. Unfortunately, the efficacy of 1,25-(OH)₂D₃ is limited by its short half-life in vivo when delivered i.p. In contrast to these studies, we demonstrated increased allograft survival in both murine non-vascularized and rat vascularized heart allografts (Hullett, et al., Transplantation 66:824-828, 1998) and here, in rat renal allografts with 1,25-(OH)₂D₃ daily in the diet. There is an important caution to consider when contemplating oral 1,25-(OH)₂D₃ therapy. While no significant hypercalcemia was observed in heart graft recipients, we did not levated serum creatinine levels and calcium deposits in the kidney tissue in some 1,25-(OH)₂D₃-treated animals. This points to a narrow therapeutic window but does suggest that if sufficient 1,25-(OH)₂D₃ can be administered without inducing hypercalcemia, then 1,25-(OH)₂D₃ may be an effective immunosuppressive agent. Van Etten, et al. have described a synergistic effect when 1,25-(OH)₂D₃ analogs were combined with CSA or mycophenolate mofitil (MMF) both in vitro and in vivo. (Van Etten et al, Transplantation 69:1932-1942, 2000). This supports our observation that 1,25-(OH)₂D₃ may have efficacy as an immunmodulatory compound.

CAN is characterized by the development of interstitial fibrosis, glomerulosclerosis, tubular atrpohy, and concentric intimal hyperplasia in arteries (transplant vascular sclerosis; Transplantation, 71:555-559, 2001; Tilney et al., Transplantation 52; 389-398, 1991). Many of these processes have been associated with the expression of TGFβ-1 (Jain et al. Transplantation 69; 1459-1766, 2000). This cytokine is a potent stimulator of ECM deposition, stimulating in kidney tissue, collagen and fibronectin synthesis by many cell types (Rasmussen et al., Am. J. Pathol. 144:1041-1048: 1995). Within the glomerulus, Nicholson, et al. have noted a specific elevation of TGFβ-1 expression following the development of CAN (Nicholson et al., Br. J. Surg. 86:1144-1148, 1999). They also noted a correlation with collagen III deposition. We have observed that he beneficial effects of 1,25-(OH)₂D₃ treatment is in part the prevention of histological changes associated with CAN. Immunostaining of 1,25-(OH)₂D₃-treated allografts showed reduced expression of TGFβ-1 in the glomeruli consistent with the prevention of proteinuria. Together with the observed decrease in glomerular collagen deposition in 1,25-(OH)₂D₃-treated allografts, these data suggest the 1) TGFβ-1 plays a direct role in CAN, 2) TGFβ-1 potentially could be used as a surrogate marker for CAN and 2) 1,25-(OH)₂D₃ modulates TGFβ-1-mediated fibrotic events.

There is an important interaction between the TGFβ-1 and the 1,25-(OH)₂D₃ signaling pathways (Yanagisawa et al. Science 283:1317-1321, 1999; Yanagi et al., J. Biol. Chem. 274: 12971-12974, 1999; Subramaniam et al. J. Biol. Chem. 276:15741-15746, 1001). Yanagisawa, et al. have demonstrated in vitro that Smad-3 functions as a coactivator to the VDR forming a heteodimeric complex with Smad-3/Smad-4 in cells over expressing VDR. We have observed complex formation between the VDR and Smad-3 in renal graft cell lysates derived from 1,25-(OH)₂D₃-treated animals suggesting an in vivo interaction as well. In addition, we and others have shown that the VDR interacts with Smad-7 both in vitro (Yanagi et al.) and in vivo. Following 1,25-(OH)₂D₃ treatment we observed a dramatic reduction in the receptor-regulated Smad 2 expression with minimal change in Smad 3 levels. One possible mechanism is that complex formation of VDR with Smad 2 may signal ubiquination and degradation. In contrast, inhibitory Smad 7 expression was increased. This may result from a TGFβ-1 auto-feedback loop or stabilization of Smad 7 expression as a VDR complex.

The changes in Smad 2 protein expression that we observed are consistent with recent finding of Li, et al. describing TGFβ-1-mediated fibrotic changes in a renal tubular epithelial cell line (Li et al., J. Am. Soc. Nephol 13: 1464-1472, 2002). Interestingly, the effects of TGFβ-1 in this system were mediated by Smad 2. Taken together, these data suggest that 1,25-(OH)₂D₃-mediated reduction in Smad 2 protein expression may be an important mechanism in preventing CAN. In contrast to the decrease in Smad 2 protein expression we observed an increase in mRNA expression. This may reflect an attempt by the kidney tissue to compensate for the dramatic loss of Smad 2 protein expression.

Progressive glomerular diseases including CAN, glomerulonephritis, diabetic nephropathy, and focal segmental glomerulosclerosis are often characterized by mesangial cell proliferation and the subsequent accumulation of ECM (Choi et al., Kidney Int. 44: 448-958, 1995). Within the glomerulus, mesangial cells in these disease settings undergro a phenotypic change (Abe et al., J. Biol. Chem. 274: 20874-20878, 1999). They up-regulate smooth muscle α-actin expression and acquire fibroblast characteristics. They also secrete collagens normally absent in the matrix e.g. collagen I and IV, in addition to secreting increased amounts of collagen III. Abe, et al. have suggested that 1,25-(OH)₂D₃ or nonhypercalcemic analogs of 1,25-(OH)₂D₃ regulate mesangial SMC phenotypes (Abe, et al. J. Biol. Chem. 274:20874-20878, 1999). Additionally, data in a 5/6 nephrectomy model and an IgA glomerulonephritis model demonstrated that 1,25-(OH)₂D₃ treatment prevented the development of glomerulosclerosis (Schwarz, et al. Kidney Int. 53:1696-1705, 1998). Strikingly, we observed decreased glomerular collagen deposition and inhibition of proteinuria in 1,25-(OH)₂D₃-treated allograft recipients. In contrast to allogeneic control grafts, these grafts showed almost no glomerular bioactive TGFβ-1 expression by immunostaining. Recent studies by Li, et al. and Chen, et al. have suggested that TGFβ-1-mediated changes in mesangial cell phenotype and collagen synthesis can be blocked by increased expression of Smad 7. (Li et al, J. Am. Soc. Nephol 13:1464-1472, 2002; Chen et al., J. Am. Soc. Nephol 13:887-893, 2002. We observed increased Smad 7 expression in 1,25-(OH)₂D₃-treated recipients. Taken together these data suggest a potential pathway for preservation of glomerular structure and function 1,25-(OH)₂D₃.

The matrix metalloproteinases are a family of proteins that are responsible for the remodeling of ECM (Johnson, et al., Curr. Opin. Chem. Biol. 2:446-471, 1998). Their expression is regulated, in part, through the Smad family of transcription factors. In concert with the TIMPs, they regulate aspects of matrix deposition and uptake. We have previously demonstrated changes in MMP and TIMP expression with CAN in transplant patients with biopsy-proven CAN (Becker, et al., Transplantation 69:1485-1491, 2000). In these studies we observed increased of MMP 2 and decreased TIMP expression 1 in 1,25-(OH)₂D₃ treated recipients. Thus, one possible mechanism by which 1,25-(OH)₂D₃ mitigates CAN is by altering the MMP/TIMP balance via VDRs known ability to form complexes with the Smad proteins.

Alternatively, VDR may act as transcription factor to directly regulate MMP/TIMP expression. Within the cell, free 1,25-(OH)₂D₃ traverses the cytoplasmic membrane where it binds the VDR. Binding of 1,25-(OH)₂D₃ to the VDR results in phosphorylation of the VDR and the ability to bind specific DNA sequences wither as a homodimer or heterodimer with the retinoid X receptor (RXR) (Darwish and DeLuca, Prog Nucleic Acid Res Mol Biol 53; 321-344:1996). VDR or VDR-RXR binding to response elements may differentially affect the expression of various genes, depending on cellular phenotype, cell cycle, and cellular activation (Strugnell and DeLuca Proc Soc Exp Biol Med 215: 223-228, 1997).

In addition to its well known role in Ca metabolism, 1,25-(OH)₂D₃, regulates immune responses, preventing the development of several autoimmune diseases in mouse models (Cantorna, et al. J. Nut 128: 68-72, 1998; Cantorna, et al. Proc Natl Acad Sci USA 93: 7861-7864, 1996). 1,25-(OH)₂D₃ addition to mixed lymphocyte cultures in vitro inhibits cell proliferation and cytotoxic T cell function (Lemire J. Steroid Biochem Mol. Biol 53: 599-602, 1995). D'Ambrosio, et al. have shown that 1,25-(OH)₂D₃ blocks interleukin 12 expression in macrophages and dendritic cells by preventing NF-κB activation and by repression of the p40 promoter (D'Ambrosio, et al., J. Clin Invest. 101: 252-262, 1998). Repression of the promoter requires binding of VDR. Recent studies also demonstrated that 1,25-(OH)₂D₃ blocks dendritic cell maturation (Griffin, et al., Pro Natl Acad Sci USA 98: 6800-6802, 2001; Berev, et al., Exp Hematol 28: 575-583, 2000). It is suggested that presentation of antigen by immature dendritic cells leads to the development of regulatory CD4+ T cells (Gregori, et al., J. Immunol 167: 1945-1953, 2001). Other studies suggest that 1,25-(OH)₂D₃ influences both Th1 an Th2 development from naive T0 cells by preventing cytokine expression (Boonstra, et al., J. Immunol 167:4974-4980, 2001). Finally, targeted disruption of functional 25-hydroxyvitamin D 1α-hydroxylase expression in mice leads to immune dysfunction (Panda, et al., Proc Natl AcadSci USA 98: 7498-7503, 2001). Thus, in addition to the effects we describe here on TGFβ-1, Smad and MMP/TIMP expression, 1,25-(OH)₂D₃ treatment may also influence the development of CAN by altering the immune environment.

Nevertheless, the discrete tissue-specific effects we have described suggest that exogenous 1,25-(OH)₂D₃ may have a direct role in regulating matrix deposition in CAN. In addition, if adequate immunosuppression could be achieved with an adjunct agent, 1,25-(OH)₂D₃ could also ameliorate aspects of transplant-related bone disease is a significant complication in transplantation and in end stage renal disease in general.

1. A method of treating chronic allograft nephropathy in transplant patients comprising the step of treating a kidney transplant patient, where in transplant patient is undergoing immunosuppressive therapy, with a sufficient amount of a vitamin D compound whereby kidney function stabilizes and wherein development of interstitial fibrosis is inhibited.

2. The method of claim 1 wherein the vitamin D compound is 1,25-dihydroxyvitamin D3.

3. The method of claim 1 wherein the vitamin D compound is are 1α-hydroxy compounds.

4. The method of claim 1 wherein the amount of vitamin D compound administered is between 0.1 μg and 50 82 g per day per 160 pound patient.

5. The method of claim 1 wherein the amount of vitamin D analog administered is between 0.1 μg and 0.75 μg per day per 160 pound patient.

6. The method of claim 1 wherein the vitamin D compound administered is administered orally and daily.

7. The method of claim 1 wherein the administration of the vitamin D compound begins within 24 hours of the kidney transplant procedure.

8. The method of claim 1 wherein the administration of vitamin D compound begins before the kidney transplant procedure.

9. The method of claim 1 wherein the administration of vitamin D compound begins after the kidney transplant procedure.

10. The method of claim 9 wherein the administration begins at least 1 year after the transplant procedure.

11. The method of claim 1 wherein the vitamin D compound is of the formula: 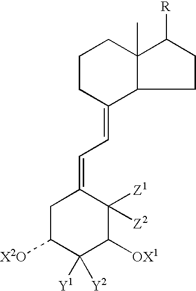
wherein X1 and X2 are each selected from the group consisting of hydrogen and acyl;
wherein Y1 and Y2 can be H, or one can be 0-aryl, 0-alkyl, alkyl of 1-4 carbons, taken together to form an alkene having the structure of 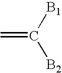
where B1 and B2 can be selected from H, alkyl of 1-4 carbons and aryl or alkyl and can have a β or α configuration;
Z1=Z2=H or Z2 together are ═CH2; and
wherein R is an alkyl, hydroxyalkyl or fluoroalkyl group, or R may represent the following side chain: 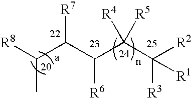
wherein (a) may have an S or R configuration, R1 represents hydrogen, hydroxy or O-acyl, R2 and R3 are each selected from the group consisting of alkyl, hydroxyalkyl and fluoralkyl, or, when taken together represents the group-(CH2)m-wherein m is an integer having a value of from 2 to 5, R4 is selected from the group consisting of hydrogen, hydroxy, fluorine, O-acyl, alkyl, hydroxyalkyl and fluoralkyl, wherein if R5 is hydroxyl or fluoro, R4 must be hydrogen or alkyl, R5 is selected from the group consisting of hydrogen, hydroxy, fluorine, alkyl, hydroxyalkyl and fluoralkyl, or R4 and R5 taken together represent double-bonded oxygen, R6 and R7 taken together form a carbon-carbon double bond, R8 may be H or CH3, and wherein n is an integer having a value of from 1 to 5, and wherein the carbon at any one of positions 20, 22, or 23 in the side chain may be replaced by an O, S, or N atom.

12. The method of claim 10 wherein the compound is selected from the group consisting of 1,25-dihydroxyvitamin D3, 19-nor-1,25-dihydroxyvitamin D2, 19-nor-21-epi-1,25-dihydroxyvitamin D3, 1,25-dihydroxy-24-homo-22-dehydro-22E vitamin D3, and 19-nor-1,25-dihydroxy-24-homo-22-dehydro-22E-vitamin D3.

13. A method of decelerating the loss of kidney function after a transplant, comprising the step of treating a kidney transplant patient, wherein the patient is undergoing immunosuppressive therapy, with a sufficient amount of a vitamin D compound wherein the loss of kidney function is decreased and wherein the development of interstitial fibrosis is inhibited.

14. The method of claim 13 wherein the vitamin D compound is 1,25-dihydroxyvitamin D3.

15. The method of claim 13 wherein the vitamin D compound are 1α-hydroxy compounds.

16. The method of claim 13 wherein the amount of vitamin D compound administered is between 0.1 μg and 50 μg per day per 160 pound patient.

17. The method of claim 13 wherein the amount of vitamin D analog administered is between 0.1 μg and 0.75.

18. The method of claim 13 wherein the vitamin D compound administered is administered orally and daily.

19. The method of claim 13 wherein the administration of the vitamin D compound begins within 24 hours of the kidney transplant procedure.

20. The method of claim 13 wherein the administration of vitamin D compound begins before the kidney transplant procedure.

21. The method of claim 13 wherein the administration of vitamin D compound begins after the kidney transplant procedure.

22. The method of claim 21 wherein the administration begins at least 1 year after the transplant procedure.

23. The method of claim 13 wherein the vitamin D compound is of the formula: 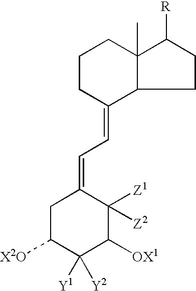
wherein X1 and X2 are each selected from the group consisting of hydrogen and acyl;
wherein Y1 and Y2 can be H, or one can be 0-aryl, 0-aryl, alkyl of 1-4 carbons, taken together to form an alkene having the structure of 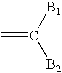
where B1 and B2 can be selected from H, alkyl of 1-4 carbons and aryl or alkyl and can have a β or α configuration;
Z1=Z2=Z or Z1 and Z2 together are ═CH2; and
wherein R is an alkyl, hydroxyalkyl or fluoroalkyl group, or R may represent the following side chain: 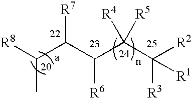
wherein (a) may have an S or R configuration, R1 represents hydrogen, hydroxy or O-acyl, R2 and R3 are each selected from the group consisting of alkyl, hydroxyalkyl and fluoralkyl, or, when taken together represents the group-(CH₂)m-wherein m is an integer having a value of from 2 to 5, R⁴ is selected from the group consisting of hydrogen, hydroxy, fluorine, O-acyl, alkyl, hydroxyalkyl and fluoralkyl, wherein if R⁵ is hydroxyl or fluoro, R⁴ must be hydrogen or alkyl, R⁵ is selected from the group consisting of hydrogen, hydroxy, fluorine, alkyl, hydroxyalkyl and fluoralkyl, or R⁴ and R⁵ taken together represent double-bonded oxygen, R⁶ and R⁷ taken togeter form a carbon-carbon double bond, R⁸ may be H or CH₃, and wherein n is an integer having a value of from 1 to 5, and wherein the carbon at any one of positions 20, 22, or 23 in the side chain may be replaced by an O, S, or N atom.

24. The method of claim 23 wherein the compound is selected from the group consisting of 1,25-dihydroxyvitamin D3, 19-nor-1,25-dihydroxyvitamin D2, 19-nor-21-epi-1,25-dihydroxyvitamin D3, 1,25-dihydroxy-24-homo-22-dehydro-22E vitamin D3, and 19-nor=1,25-dihydroxy-24-homo-22-dehydro-22E-vitamin D3.
